(19) United States
(12) Patent Application Publication
(10) Pub. No.: US 2008/0213824 A1
(43) Pub. Date: Sep. 4, 2008

(54) Synthesis of Labeled Probes

(75) Inventor: Ilia Ichetovkin, Hamden, CT (US)

Correspondence Address: KELLEY DRYE & WARREN LLP, 400 ALTLANTIC STREET , 13TH FLOOR, STAMFORD, CT, 06901, US

(73) Assignee: IKONISYS, INC., NEW HAVEN, CT (US)

(21) Appl. No.: 11/833,365

(22) Filed: Aug. 3, 2007

Related U.S. Application Data
(60) Provisional application No. 60/821,555, filed on Aug. 4, 2006.

(51) Int. Cl. G01N 33/483; C07K 2/00; C07H 21/00; C07H 21/04

(52) U.S. Cl. 435/40.5; 530/300; 536/22.1; 536/24.3

(57) ABSTRACT

Improvements in FISH staining employing an aqueous acetonitrile/dimethylsulfoxide mixture in a fluorophore coupling reaction.

### CROSS REFERENCE TO RELATED APPLICATIONS

This application claims the benefit of priority of U.S. Provisional Application No. 60/821,555 filed Aug. 4, 2006 (Atty. Docket No. IK-165(P)), which is incorporated herein in its entirety.

### BACKGROUND OF THE INVENTION

All references cited in this specification, and their references, are incorporated by reference herein where appropriate for teachings of additional or alternative details, features, and/or technical background.

### FIELD OF THE INVENTION

The present invention generally relates to an apparatus and method for improving FISH staining, in particular when a hydrophobic fluorescent tag is employed.

### DESCRIPTION OF THE RELATED ART

Many methods are known to aid in the microscopic analysis of samples. For example, without limitation, it is known that certain dyes have an affinity for certain cellular structures. Such dyes may therefore be used to aid in analysis by helping to further elucidate such structures.

Fluorescence microscopy of cells and tissues is well known in the art. Treating cells with fluorescent reagents and imaging the cells is well known in the art. Methods have been developed to image fluorescent cells in a microscope and extract information about the spatial distribution and temporal changes occurring in these cells. Some of these methods and their applications are described in an article by Taylor, et al. in American Scientist 80 (1992), p. 322-335. These methods have been designed and optimized for the preparation of a few specimens for high spatial and temporal resolution imaging measurements of distribution, amount and biochemical environment of the fluorescent reporter molecules in the cells. Detection of fluorescent signals may be by way of an epifluorescent microscope which uses emitted fluorescent light to form an image (whereas a conventional reflecting microscope uses scattered illumination light to form an image). The excitation light of a epifluorescence microscope is used to excite a fluorescent tag in the sample causing the fluorescent tag to emit fluorescent light. The advantage of an epifluorescence microscope is that the sample may be prepared such that the fluorescent molecules are preferentially attached to the biological structures of interest thereby allowing identification of such biological structures of interest.

The acronym “FISH” references a technique that uses fluorescein tags that glow under ultraviolet light to detect chromosomal structure. FISH uses fluorescent probes which bind only to those parts of the chromosome with which they show a high degree of sequence similarity. Such tags may directed to specific chromosomes and specific chromosome regions. The probe has to be long enough to hybridize specifically to its target (and not to similar sequences in the genome), but not too large to impede the hybridization process, and it should be tagged directly with fluorophores. This can be done in various ways, for example nick translation and PCR using tagged nucleotides. If signal amplification is necessary to exceed the detection threshold of the microscope (which depends on many factors such as probe labelling efficiency, the kind of probe and the fluorescent dye), fluorescent tagged antibodies or streptavidin are bound to the tag molecules, thus amplifying the fluorescence.

The FISH technique may be used for identifying chromosomal abnormalities and gene mapping. For example, a FISH probe to chromosome 21 permits one to “fish” for cells with trisomy 21, an extra chromosome 21, the cause of Down syndrome. FISH kits comprising multicolor DNA probes are commercially available. For example, AneuVysion Multicolor DNA Probe Kit sold by the Vysis division of Abbott Laboratories, is designed for in vitro diagnostic testing for abnormalities of chromosomes 13, 18, 21, X and Y in amniotic fluid samples via fluorescence in situ hybridization (FISH) in metaphase cells and interphase nuclei. The AneuVysion® Assay (CEP 18, X, Y-alpha satellite, LSI 13 and 21) Multi-color Probe Panel uses CEP 18/X/Y probe to detect alpha satellite sequences in the centromere regions of chromosomes 18, X and Y and LSI 13/21 probe to detect the 13q14 region and the 21q22.13 to 21q22.2 region. The combination of colors evidenced is used to determine whether there is normal chromosome numbers or trisomy. In a similar vein, the UroVysion kit by the Vysis division of Abbott Laboratories designed to detect chromosomal abnormalities associated with the development and progression of bladder cancer by detecting aneuploidy for chromosomes 3, 7, 17, and loss of the 9p21 locus via fluorescence in situ hybridization (FISH) in urine specimens from persons with hematuria suspected of having bladder cancer.

Incorporation of certain fluorescent labels into nucleotides, or the labeling of a polynucleotide using a labeled monomer, may be problematic in cases where the label is excessively hydrophobic and fails to dissolve in a reaction medium. In such cases, when conducting a synthetic reaction, or when used in a FISH protocol, the label or a label-derivatized moiety, may precipitate. This leads to poor reaction yields, or to spurious staining results in a sample or specimen. The present invention addresses these and related concerns.

### SUMMARY OF THE INVENTION

Embodiments disclosed herein include:

A method of preparing a labeled biomolecule comprising (a) suspending a labeling reagent comprising a first reactant moiety in a solubilizing composition to achieve a solution comprising said labeling reagent in said composition; (b) contacting a biomolecule bearing a second reactant moiety reactive with the first reactant moiety with the solution comprising said labeling reagent under conditions that promote reaction between the first reactant moiety with the second reactant moiety; thereby labeling the biomolecule.

In various embodiments wherein the biomolecule is a polypeptide, or a nucleic acid, or a probe nucleic acid. In various embodiments the probe specifically hybridizes with a target nucleic acid sequence.

In various embodiments the second reactant moiety comprises a carboxylate, or the second reactant moiety comprises an amino group.

In various embodiments the first reactant moiety reacts with a carboxylate group, or the first reactant moiety reacts with an amino group, or the first reactant moiety comprises a carboxylate succinimidyl ester.

In various embodiments the label is a fluorescent label, or the label comprises a diethylaminocoumarin moiety. In additional embodiments the biomolecule comprises a probe nucleic acid.

In various embodiments the solubilizing composition comprises acetonitrile, dimethylsulfoxide and water; in various embodiments the acetonitrile is present in a proportion of about 2% (v/v) to about 50% (v/v, or the dimethylsulfoxide is present in a proportion of about 10% (v/v) to about 50% (v/v), or the water is present in a proportion of about 20% (v/v) to about 50% (v/v).

In additional embodiments there is disclosed a solution comprising a derivative of a hydrophobic fluorescent label in a solubilizing composition. In various embodiments the fluorescent probe comprises a diethylaminocoumarin moiety. In additional embodiments the solubilizing composition comprises acetonitrile, dimethylsulfoxide and water.

### DETAILED DESCRIPTION OF THE INVENTION

When highly hydrophobic fluorescent tags are being used in the FISH protocol, it has been found that precipitation of the fluorescent tag may be a problem when conventional solvent systems are used during the coupling reaction. It has been surprisingly found that an aqueous mixture of acetonitrile and dimethylsulfoxide (DMSO) can be used to improve yields of fluorophore coupling to a biomolecule and prevent FISH probe contamination during the labeling process. Advantageously a composition containing a FISH probe in a mixed aqueous solvent of acetonitrile and DMSO, where the ratios of the acetonitrile may be present in a proportion of about 2% (v/v) to about 50% (v/v); the dimethylsulfoxide may be present in a proportion of about 10% (v/v) to about 50% (v/v); the water may be present in a proportion of about 20% (v/v) to about 50% (v/v). In these solvent mixtures, a mixture having any ratio of the cosolvents within the stated range limits is contemplated.

A biomolecule that may be labeled for use in a labeled diagnostic or assay procedure includes a nucleic acid; a polynucleotide; an oligonucleotide; any derivative of a nucleic acid, polynucleotide or oligonucleotide wherein the phosphodiester grouping is substituted with related groupings that confer ease of synthetic preparation and/or greater stability in biological or assay conditions; a further derivative of nucleic acid, polynucleotide, oligonucleotide, or probe nucleic acid derivatized to include a second reactive moiety that is specifically reactive with a first reactive moiety on a labeling reagent; a protein; a polypeptide; an oligopeptide; and the like. Nonlimiting examples of a second reactive moiety on a probe nucleic acid include an aminoallyl group or a 5-aminohexylacrylamido group. Additionally a second reactive moiety may comprise a carboxylate group. Nonlimiting examples of a first reactive moiety bound to a labeling reagent include a reactive moiety that targets pendant amino groups, or that targets pendant carboyxlate groups. Labeling chemistries and procedures are widely known to workers of skill in the field of the invention, and are available for example from Pierce Chemical Co., Rockford, Ill.

In an exemplary procedure for preparing a fluorescently labeled nucleic acid probe useful in a FISH analysis, a solubilizing composition is prepared using 2% to 50% (v/v) acetonitrile, 10%-50% dimethylsulfoxide (v/v), and 20%-50% water (v/v). The solubilizing composition is buffered at about ph 8.0-9.0 to promote the reaction between the reactant moieties, for example by including 0.1%-5% NaHCO3 or Sodium Borate (Na2[B4O5(0H)4].8H₂O). This composition is used to prepare solution of a reactive derivative of a fluorescent label, 7-diethylaminocoumarin-3-carboxylic acid succinimidyl ester (available from Anaspec Corp., San Jose, Calif.) at a concentration of about 0.1 ug/ul to 50 ug/ul. This solution of the fluorescent label is combined with a solution containing a probe DNA molecule which is derivatized to incorporate pendant aminoallyl groups, or pendant 5-aminohexylacrylamido groups. These groups can provide a second reactant moiety that is an amino group for specific reaction with carboxylate succinimidyl ester of the fluorescent label. Upon incubation for sufficient time under suitable conditions for reaction to proceed, the probe DNA is labeled with diethylaminocoumarin. The product is useable in a FISH analysis.

### STATEMENT REGARDING PREFERRED EMBODIMENTS

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

What is claimed is:

1. A method of preparing a labeled biomolecule comprising
(a) suspending a hydrophobic labeling reagent comprising a first reactant moiety in a solubilizing composition containing water to achieve a non-precipitating solution comprising said labeling reagent in said composition;
(b) contacting a biomolecule bearing a second reactant moiety reactive with the first reactant moiety with the solution comprising said labeling reagent under conditions that promote reaction between the first reactant moiety with the second reactant moiety; thereby labeling the biomolecule.

2. The method of claim 1 wherein the biomolecule is a polypeptide.

3. The method of claim 1 wherein the biomolecule is a nucleic acid.

4. The method of claim 1 wherein the biomolecule is a probe nucleic acid

5. The method of claim 3 wherein the probe specifically hybridizes with a target nucleic acid sequence.

6. The method of claim 1 wherein the second reactant moiety comprises a carboxylate.

7. The method of claim 1 wherein the second reactant moiety comprises an amino group.

8. The method of claim 1 wherein the first reactant moiety reacts with a carboxylate group.

9. The method of claim 1 wherein the first reactant moiety reacts with an amino group.

10. The method of claim 1 wherein the first reactant moiety comprises a carboxylate succinimidyl ester.

11. The method of claim 1 wherein the label is a fluorescent label.

12. The method of claim 1 wherein the label comprises a diethylaminocoumarin moiety.

13. The method of claim 12 wherein the biomolecule comprises a probe nucleic acid.

14. The method of claim 1 wherein the solubilizing composition comprises acetonitrile, dimethylsulfoxide and water.

15. The method of claim 14 wherein the acetonitrile is present in a proportion of about 2% (v/v) to about 50% (v/v).

16. The method of claim 14 wherein the dimethylsulfoxide is present in a proportion of about 10% (v/v) to about 50% (v/v).

17. The method of claim 14 wherein the water is present in a proportion of about 20% (v/v) to about 50% (v/v).

18. A solution comprising a derivative of a hydrophobic fluorescent label in a solubilizing composition.

19. The solution described in claim 18 wherein the fluorescent probe comprises a diethylaminocoumarin moiety.

20. The solution of claim 18 wherein the solubilizing composition comprises acetonitrile, dimethylsulfoxide and water.

21. The solution of claim 20 wherein the acetonitrile is present in a proportion of about 2% (v/v) to about 50% (v/v).

22. The solution of claim 20 wherein the dimethylsulfoxide is present in a proportion of about 10% (v/v) to about 50% (v/v).

23. The solution of claim 20 wherein the water is present in a proportion of about 20% (v/v) to about 50% (v/v).
